(19) United States
(12) Patent Application Publication
(10) Pub. No.: US 2021/0322248 A1
(43) Pub. Date: Oct. 21, 2021

(54) WEARABLE ACTIVE ASSISTING DEVICE

(71) Applicant: MYOSWISS AG, Zurich (CH)

(72) Inventors: Alejandro Sancho PUCHADES, Zurich (CH); Kai SCHMIDT, Zurich (CH)

(21) Appl. No.: 17/272,523

(22) PCT Filed: Sep. 6, 2019
(86) PCT No.: PCT/EP2019/073863

§ 371 Date: Mar. 1, 2021

(30) Foreign Application Priority Data
Sep. 6, 2018 (DE) 10 2018 215 154.7

(51) Int. Cl. A61H 1/02; G06F 1/16; A61B 5/11

(52) U.S. Cl. CPC A61H 1/0237; A61H 2201/165; A61B 5/1124; G06F 1/163

(57) ABSTRACT

The present invention relates to a wearable active assisting device comprising an actuator in use to provide a limb assistance and coupled to a limb to be actively assisted via at least one force transmission element to be elongated or shortened by the actuator; and a control having an input for signals from a plurality of sensors, a signal processing stage for processing input signals from the plurality of sensors, and an output stage for outputting a motor actuation signal in accordance with the processed sensor signals; wherein the control further has a limb assistance degree selection input for selecting a degree of limb assistance; and wherein the signal processor stage is adapted to continuously model an elongation of the at least one force transmission element to be elongated or shortened corresponding to a movement or posture currently detected by the plurality of sensors to output a continuous actuator actuation signal according to a modeled elongation of the at least one force transmission element to be elongated or shortened and in response to a selected minimum degree of limb assistance.

The present invention relates to a wearable active assisting device.

Wearable active assisting devices are well-known. They can be used in particular for assisting a patient that is impaired in his or her movements, for example due to an accident, due to a recent surgery or due to another medical condition. Wearable active assisting devices both help the patient to move in a manner at least close to normal and may also be used to help the user train moving in a normal manner without assisting device. This is done not only by actively assisting in movement of limbs but also by providing external support and stability.

When a patient recovers or prior to deterioration of a patient's health in case of progressing diseases, the assistance provided by the device may not require the full power possible for a given active assisting device. Frequently, either certain limbs need not be assisted at all or need not be assisted to the full extent. In particular, for example during training sessions of a patient recovering from an accident, it may be helpful to gradually decrease assistance or to reduce overall assistance to zero. This, however, in standard wearable active assisting devices is difficult.

In the document “Smart Suit for Horse Trainers—Power and Skill Assist Based on Semi-active Assist and Energy Control”, International Conference on Advanced Intelligent Mechatronics, Montréal, Canada, Jul. 6-9, 2010, by T. Kusaka et al., inspect accession number 11 769922, a power assist system is suggested wherein force generated by an elastic material is controlled by adjusting its stretch or offset. It is suggested to synchronize user movement and assist force and to this end, it is suggested to apply a periodical input control method wherein periodical movement is to change with equine movement and wherein the length of the elastic material is adjusted to synchronize with the period of equine movement so as to obtain suitable assistance.

US 2018/0078391 describes walking assistance based on estimated joint torques that requires electromyogram (EMG) data and motion data of the user as input. Based on the different estimated joint torques, parameters of the device will be set for locomotion, which are particularly mimicking the torques in the joints generated by the human body. A device known from DE 10 2012 219 429 A1 is controlled by measuring the remaining energy of the actuators and used together with a remainder detector to determine the degree of assistance.

From WO 2018/122106 a soft wearable muscle assisting device is known wherein tendons are shortened or lengthened or maintained with respect to the length and position using a DC motor to which a control signal is provided. A controller may use an array or a plurality of motion and force sensors used in a manner that estimates the user's postures and/or movement intentions or current movement. Based on this information, the controller of the apparatus may decide how to optimally support the user's movement e.g. by modulating the forces applied and the stiffness of the joints. It is suggested that the sensor setup may include inertial-measurement units at the shank and thigh segments of the leg to measure leg kinematics, of the arms to measure arm kinematics and of the body's center of mass to measure trunk movements. Also, load cells are suggested to be placed at each tendons to measure forces. Encoders in the motors are suggested to continuously measure the rotational position of the motor shaft of the actuators, thus estimating the length of the tendons. It is stated that the combination of the load cells and encoders and/or the encoder's signals alone allow fine control of the stiffness and/or the force levels in the system. It is also stated that a motor can apply forces equivalent to the influence of gravity and to modulate joint stiffness.

Wearable active devices, known from WO/2016/089466, WO/2015/157731 and WO/2018/039354 that rely on cables to provide assistive forces, are not able to provide minimal assistance or to closely follow the movement of the wearer. When no force is needed, these devices switch into a mode where there is enough slack in the force transmitting cable to allow the user to execute the full range of motion without restriction. Thus, these systems are not able to transmit forces immediately when needed unexpectedly since they have to first overcome the excessive slack. This does not only reduce the bandwidth of these systems significantly, but also does not allow a smooth onset of force transmission. Furthermore, this principle is also less energy efficient because it requires the actuators to actively feeding out additional the cable to generate enough slack.

While some of the known wearable active assisting devices, in particular the soft wearable muscle assisting device known from WO2018/122106 A1, provide very good assistance to a user, it would be desirable to allow selection of the degree to which a limb is assisted or a plurality of limbs are assisted, even if the desired degree of assistance is, at least for some of the time, zero or neglectable for the user. Also, providing a support actually needed more exactly would be appreciated by many users.

It is an object of the present invention to provide novelties for industrial application.

This object is achieved by the subject matter of the independent claim. Some of the preferred embodiments are claimed in dependent claims.

According to a first basic idea of the present invention, a wearable active assisting device comprising a motor actuatable in use to provide a limb assistance and coupled to a limb to be actively assisted via at least one force transmission element to be elongated or shortened by the motor; and a control having an input for signals from a plurality of sensors, a signal processing stage for processing input signals from the plurality of sensors, and an output stage for outputting a motor actuation signal in accordance with the processed sensor signals; is suggested wherein the control further has a limb assistance degree selection input for selecting a degree of limb assistance; and wherein the signal processor stage is adapted to model an elongation of the at least one force transmission element to be elongated or shortened corresponding to a movement currently detected by the plurality of sensors and to output a motor actuation signal according to a current modeled elongation of the at least one force transmission element to be elongated or shortened and in response to a selected minimum degree of limb assistance.

In other words, a wearable active assisting device is suggested having a selectable minimum degree of assistance, which can be close to zero limb assistance, by actuating the motors usually assisting the limb in a manner so that the elongation of the force transmission element is tightly controlled according to a model derived based on the plurality of sensor signals. This allows selecting minimum assistance without decoupling the actuators, motors and the like from of the tendons. In particular, it is possible that the minimum degree of limb assistance is selected not by the user wearing the wearable active assisting device himself but by a physical therapist, physiotherapist, medical doctor and so forth, in particular even without the patient noticing. As the motor continues to elongate or shorten at least one force transmission element, even where no actual support is provided, a user hearing the motor will have the impression of being supported.

Thus, a placebo effect of the wearable active assisting device can easily be tested, in particular where a patient has to rebuild confidence in his (or her) own muscles. What is more is that by modeling elongation and by shortening and/or elongating the at least one force transmission element, in case where it turns out that assistance must still be provided for certain movements in spite of the expectation of the physiotherapist or the like, assistance can and will immediately be available. It should be noted that for the purpose of the present invention, a limb assistance degree selection input is adapted so that with the unit switched on at least 2 different degrees of support are selectable, the minimum degree of limb assistance being one of these degrees selectable even where this minimum degree corresponds to zero assistance.

It is noted that a different degree of assistance can be selected in preferred embodiments for both sides of a human body, for example an assistance to the left leg being different from the assistance to the right leg. Where separate actuators are used to assist limbs on the same side of the body, it may also be possible and advantageous to select a different degree of assistance for each limb. It is noted that the output of the motor-actuated signal may be modeled by simply referring to certain signals indicating for example the current posture, that is the flexing or bending state of the limb to be assisted and, where the force transmission element runs further than across one joint of the limb to be assisted to the motor, for example because simultaneous assistance with one electronic motor is to be provided to the shank, the thigh and the hip by a force transmission element adequately guided, it is possible to only rely on sensor signals measuring the angle of the joints, the orientation of the respective element, judging for example whether the thigh is in a vertical, horizontal or intermediate position and so forth. It is not necessary to determine whether a person currently requiring minimum, preferably zero assistance for a specific limb is moving according to a specific pattern such as walking, climbing stairs and so forth.

Thus, it is neither necessary nor considered particularly advantageous to predict a next movement in order to provide the minimum limb assistance. The elongation or shortening of the force transmission element by the motor actuated by the motor-actuation signal controlled as suggested by the present invention need not rely on a predefined position trajectory but can, and preferably will, be continuously scaled according to one or more processed sensor signals, so that a minimum limb assistance can be continuously provided regardless of the movement or posture being performed.

It is possible to nonetheless have an elongation or shortening of the at least one force transmission element that closely matches the current behavior of the user without predicting the way the user will move next. It is even advantageous to only rely on the current sensed signals from the plurality of sensors rather than rely on a predicted movement pattern.

Nonetheless, it is to be noted that even while using the wearable active assisting device in a transparency mode wherein a minimum degree of limb assistance is provided, the current movement could still be identified together with the phase of a user's current movement. In this manner, it is possible to immediately assist without delay and without adverse effects a user that all of a sudden requires assistance for example because it is detected that the blood pressure or heart rate of the user increases beyond critical levels which frequently may be due to either having the impression that one cannot cope with the current situation or because actual efforts are too large for the user.

Also, acceleration sensors and/or angular velocity sensors could indicate that the user begins to move in a falling manner and that a fall must be prevented. In such cases, detecting the current movement pattern may be helpful even though the actual elongation or shortening of the force transmission element while the system is in a minimum degree of limb assistance state will not rely on such patterns. It is noted that the control can be implemented using hardware stages such as hardware-implemented filters and the like, or that, as an alternative, the sensor signals could be conditioned and digitized so that the control can be implemented as a software stage. It is possible to include the control as an additional (software) module into pre-existing active assisting devices, in particular where such devices already provide adequate sensor signals.

In a preferred embodiment, the plurality of sensors comprises gyro- and/or accelerometer sensors and/or magnetometer sensors and/or stretchable sensors and/or kinematic and/or angle sensors. (Multiaxial, in particular triaxial acceleration sensors, gyro- and magnetometer sensors are particularly useful in determining the current orientation of a limb or limb segment. Also, providing a plurality of gyro- and/or accelerometer sensors on both sides proximal and distal of a joint allows to determine or at least estimate the angle of the joint. The same holds for magnetometer sensors that will allow to determine the orientation in the magnetic field of the earth. It should be noted that the aforementioned sensors and the associated signal conditioning circuitry such as buffers, amplifiers, A/D converters and the like may be affected by changing ambient conditions such as temperature in a predictable manner. Therefore, in certain embodiments, it might be preferred to have additional ambient sensors such as temperature sensors, barometric sensors asf. and to correct for potential drifts of the gyro- and/or accelerometer sensors and/or magnetometer sensors and/or stretchable sensors and the associated circuitry in response to signals derived from the additional ambient sensors.

It is noted that other sensors such as dedicated angle sensors for determining the angle of a joint can be used. It can be foreseen that upcoming sensors such as stretchable sensors and/or new and/or other known, though not explicitly listed kinematic and/or angle sensors are usable. However, it is particularly advantageous if no force sensors required to measure the tension on the force transmitting element, for example strain gauges and the like, as this simplifies the arrangement and reduces costs. It should be noted that even without strain gauges attached to the force transmission element, the reaction of the wearable active assisting device according to the invention can be very fast.

In a preferred embodiment, the active assisting device is adapted to assist in an activity of one or more limb, in particular at least one leg of the human body. It is possible to rely on control signals from only one leg to provide the control signals. However, it might be preferred to use signals from both legs. For example, where a user begins to fall, high accelerations are to be expected and generally these will not be assignable to any known typical behavior or movement. Thus, relying on signals from both sides will allow the system to determine faster that a zero or minimum assistance phase needs to be terminated immediately.

In a preferred embodiment, as indicated above, the control is adapted to model a force transmission element elongation independent of any force- or tension-indicative signals indicative of a measurement of a force and/or tension in the force transmission element, in particular independent of load cell sensor measurement signals. It will be understood that while in case such forces or tensions are measured anyhow, it would be possible to use the corresponding signals also in a transparency mode; however, as for most applications no such force- or tension-indicative signals are really needed either, it can be considered an advantage if no such corresponding sensor signals need to be provided as they would then serve only to implement the transparency mode. Therefore, it will also be understood that while such force or tension sensors might be used in a transparency mode, if they are not considered vital for implementing additional functionalities, they will not be needed.

In a highly preferred embodiment, the force transmission element itself will be hardly extendable. In other words, during normal use, the forces that the user may exert on the force transmission element will not be suitable to allow for a large extension thereof.

Now, as has been indicated above, the wearable active assisting device according to the present invention relies on a module for assessment of the currently needed elongation of the force transmission element by actuating the motor. In this context, it should be noted that unwinding and winding-in of a cable where the cable corresponds to the force transmission element is considered elongating or shortening the force transmission element.

It will be understood that basically modeling of the elongation will be most precise if the physical parameters such as size, length of limbs, circumference of legs and the like are well known.

However, there frequently is a need or at least a wish that the user need not be measured very precisely because this will take time, in particular the time of a physiotherapist or the like increasing overall costs of using a wearable active assisting device according to the invention.

Therefore, it is highly preferred to allow that the wearable active assisting device can be used without determining dedicated physical parameters for each single user overly exact. Therefore, in a highly preferred embodiment, between the limb and a motor, a resilient element is provided in series with the force transmission element to be elongated or shortened. Using such a resilient element, for example a coil spring, allows small errors in the determination of an elongation or shortening of the force transmission element currently needed to remain undetectable to a user.

In the preferred embodiment, it is further preferred that a restrictor is provided that limits or restricts the elongation of the resilient element, for example restricts the elongation of the spring to a maximum allowed elongation, and that takes up any additional forces else applied to the spring or other resilient element without allowing further elongation thereof. For example, a specific length of a cord or wire could be provided within a coil spring. The cord could be attached at the same points as the ends of the spring, so that the restrictor also would be placed between the limb and a motor used for assisting the limb when actuated. Given that the rope shall be longer than the spring coil as long as the spring coil is not extended, all forces exerted on the force transmission element, for example due to the mismatch between model and user, will lead to an extension of the spring to a certain degree.

While the extension of the spring remains low, no forces are taken up by the restrictor. However, once the spring is extended to an allowed maximum, any additional forces will be taken up by the restrictor and will thus not allow further extension of the resilient spring. In other words, the restrictor restricts elongation to a specific maximum allowed in particular where actual support or assistance is provided. By properly selecting an adequate maximum allowed elongation of the resilient spring element and a suitable modulus of resilience, care can be taken that any deviation between the model and an extension actually needed for a specific user will neither impair the intended behavior of the present wearable active assisting device during actual assistance nor will it render the device sensible during a transparency mode.

In a preferred embodiment, the resilient element has a modulus of resilience such that for a maximum residual force acceptable in a selected minimum degree of limb assistance, the resilient element is elongated by no more than the maximum allowed deviation between a standardized model and the correct extension for a given user. In this context, it will be obvious that despite not having to take very precise measures of a user, it is possible and will be preferred to provide a plurality of resilient elements differing both in resilience and maximum allowed length. In a typical situation, the maximum allowed deviation may be a few centimeters, for example 3 to 7 cm. This distance can easily be overcome even in case of an emergency change of limb assistance given standard motor speeds. These maximum allowable lengths that are preferred in turn allow residual forces due to the extension of the resilient element when mismatched to the model that are hardly detectable by a user and will ensure that a mismatch remains undetected most or all of the time.

As indicated above, it is possible to determine a phase in current movement identified and to output a motor actuation signal in response to a modeled elongation. While it would be possible to predict the actual extension, however, it should be noted that for the transparency mode provided by a wearable active assisting device according to the present invention, neither knowledge of for example a gait phase is needed nor does the system rely on an accurate gait cycle, since the actuation profile is not predefined. In particular, it should be noted that instead of relying on specific gait phases, other parameters such as a continuous force scaling depending on the knee angle asf. can be used.

However, nonetheless, the control may be adapted to identify certain activities such as walking, standing, walking uphill or downhill, ascending or descending a stair, sitting transitions and so forth. As indicated above, even where the control of the transparency mode itself need not rely on the determination of the precise current activity, safety of the device can be increased.

Even though the actuation in transparency mode does not rely on any predefined actuation profile corresponding to some identified mode of movement, the control may be adapted to not just identify certain activities, but to also combine the continuous force scaling actuation present in the transparency mode with predefined actuation profiles required at certain stages of a detected activity, alternating between continuous force scaling assistance and predefined actuation profiles when desired.

In a preferred embodiment, the minimum degree of assistance can be selected such that the residual force remains smaller than 30 N at the limb, preferably smaller than 20 N at the limb, in particular smaller than 10 N at the limb. However, usually the residual force will nonetheless be larger than 0.5 N, in particular larger than 1 N, in particular between 0.5 and 5 N for at least a part of a movement, in particular 50% of movement that is cyclic, preferably for at least 66% of a cyclic movement, in particular preferred for at least ¾ of a cyclic movement.

While other mechanisms could be used, in the most preferred embodiment, the at least one force transmission element will be a tendon such as a cable or a rope that for elongation thereof is reeled or unreeled using an electric motor such as a step motor or a brushless motor. The motor can in particular be a brushless motor, which is particularly preferred because, when assistance is needed, brushless motors can be easily controlled and provide sufficient high torque on a limb to be assisted. In a preferred embodiment, the force transmission element is guided in a slack sheath. In other words, the force transmission element need not be a Bowden cable or the like so that the overall construction of the wearable active assisting device is simplified.

It is noted that basically the control is adapted to model the elongation such that in transiting from a selected minimum degree of assistance to a degree of assistance higher than the minimum degree, a force transmission element slack of no more than 10 cm, preferably no more than 7 cm, in particular preferably no more than 5 cm needs to be overcome by reeling prior to providing a perceptible assistance to the user.

It will be understood that even where some slack is present in the system as the model relies on an “average user” for modeling, the resilient element will equalize model errors that usually are related to small errors. In a preferred embodiment, the specific size using a multitude of parameters need thus not be entered into the system.

In a preferred embodiment, it may be useful to guide the force transmission element such that it extends beyond more than one joint. In this manner, forces can be applied through large parts of a (cyclic) movement. Where the control is adapted to model an elongation of the at least one force transmission element to be elongated or shortened, it is highly preferred to take into account not only the actual size currently needed but also friction.

In a preferred embodiment, it is taken into account that the variable active assisting device must comprise (in order to provide active assistance at all) a plurality of garment-like elements and so forth that the user has to wear. Compared to normal clothing, such garment-like elements will currently still be significantly stiffer than conventional clothing and will also have a higher weight. When moving in such a wearable assistive device, the additional friction caused by the garment-like elements and so forth needs to be overcome, and when accelerating part of the human body, it frequently will not be sufficient to simply provide zero force to a limb. Rather, in certain cases, it is preferable that a user will not be affected at all by wearing the assisting suit. Accordingly, in a true transparency mode where assistance is zero, preventing a “negative” assistance also is desirable so that effects such as friction and inertia do not affect the user. Hence, these effects should be compensated for.

In a preferred embodiment of the wearable active assisting device, even where a torque during assistance is actively applied only in one direction, there may be antagonistic passive elements that help in stabilizing joints and so forth. If this is the case, then for a transparency mode, the antagonistic passive forces also need to be counteracted. In that case, any limb assistance will only result from a residual stabilization of joints but not from actively empowering movements, so that some assistance still is provided.

It is noted that any models implemented or used by the controller may be designed such that a tissue compliance and/or body shape of the user wearing the active assisting device is taken into account as part of the model. In other words, the user itself preferably is considered an integral part of the controller of a system that is being worn. This allows the controller to assist the user in a manner taking advantage of the compliance of the human body as if it were a spring-damper system that stores and/or attenuates, in other words, absorbs energy from the force transmitting element, thus avoiding instabilities in the control scheme, and thus, helps stabilize any potential instabilities during the control actions, ensuring a safety actuation that at the same time is capable of a sudden increases of assistance if required. Accordingly, an additional level of safety can be achieved that allows the system to assist with high levels of assistance in a sudden but controlled manner if required.

It will be understood that even where a limb assistance different from zero assistance is to be provided by the wearable active assisting device of the present invention, it may be helpful and is considered inventive per se, to model an elongation of the at least one force transmission element to be elongated or shortened corresponding to a movement or posture currently detected by the plurality of sensors and to output a motor actuation signal according to a current modeled elongation of the at least one force transmission element to be elongated or shortened and taking into account the selected degree of limb assistance. In other words, during active assistance in particular with a degree of assistance lower than the maximum assistance possible, the transparency mode can be used as a base and any actual assistance can then be combined by further elongating or shortening the at least one force transmission element so as to provide the limb assistance to the degree actually assisted. From this, it can be seen that the transparency mode can be combined with other modes of assistance allowing a continuous assistance throughout any given movement of at least the selected minimum level of assistance.

It should be noted that when referring to an actuator, not only electric motors are usable. For example, in the context of the invention, such motors could be hydraulic or pneumatic as well. Even technologies that resemble artificial muscles and allow control thereof could be used. It will be understood that devices may e.g. take advantage of electrostriction, magnetostriction and the like.

Protection is also sought for a control of a wearable active assisting device that has a motor actuatable to provide limb assistance and coupled to a limb to be actively assisted via at least one force transmission element to be elongated or shortened by the motor; the control having an input for signals from a plurality of sensors, a signal processing stage for processing the signals, and an output stage for outputting a motor actuation signal in accordance with the processed sensor signals; and wherein the control comprises a model stage adapted to model the elongation of the at least one force transmission element in a manner keeping assistance at or below a threshold sensible by the user by taking into account both a current movement and/or posture of the user detected by the sensors, the compliance of the tissue of the human body and an inertia and/or friction of the wearable active assisting device counteracting a movement, and wherein the output stage is adapted to output the motor actuation signal in accordance with the current modeled elongation and a limb assistance required.

In this control, applying the same general ideas of the invention, an influence of the wearable assistive device hardly noticeable by a user can be obtained even without adjusting the level of support to a minimum by instead taking into account inertia and/or friction of components so that these can be compensated. From this, it will be obvious that even where no minimum assistance is required, the transparency mode is helpful in increasing the precision with which a given amount of assistance can be reached and this can be done by using a minimum (=transparent) mode as a base line. As can be understood, this can be done by taking also into account at least one of friction and/or inertia components even where no minimum assistance is required and by then applying the concepts disclosed.

The present invention will now be explained with reference to the drawings. In the drawings:

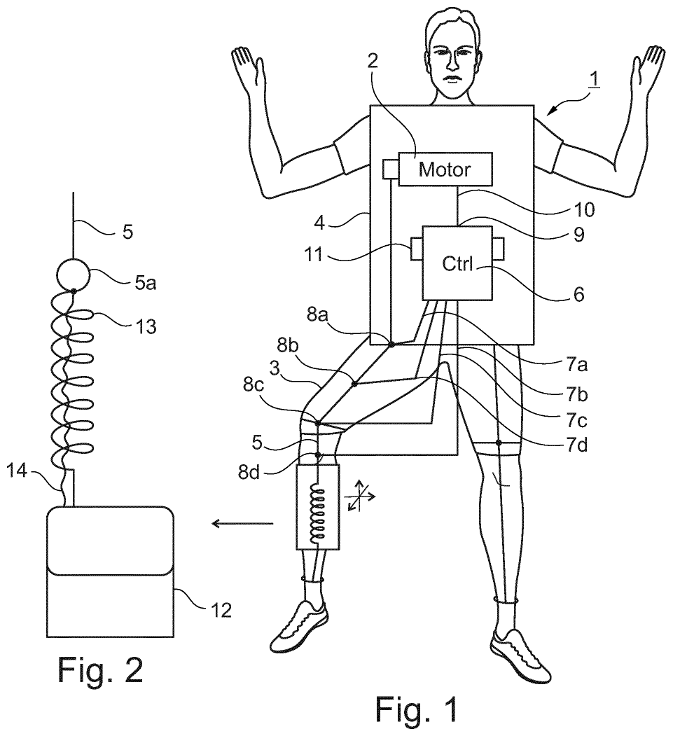

FIG. 1 shows a schematic of a wearable active assisting device according to the present invention;

FIG. 2 shows a detail thereof showing a spring as resilient elastic element provided in series with a force transmission element to be elongated or shortened and a cuff arrangement to be placed around a limb but together with a restrictive for restricting the spring elongation to the maximum allowed elongation;

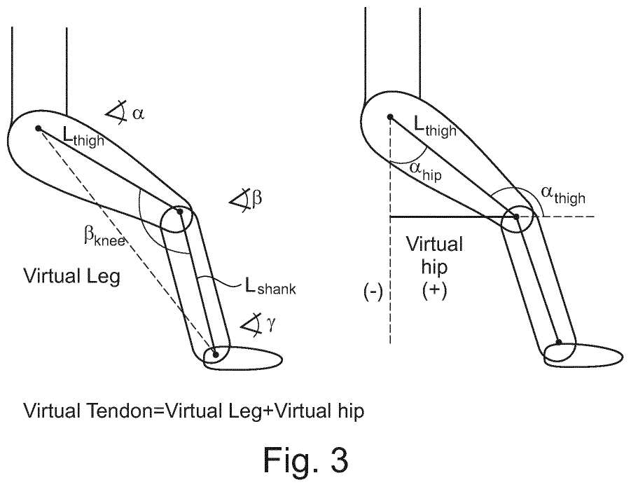

FIG. 3 shows an explanation of a model used by the control of the wearable active assisting device according to the invention;

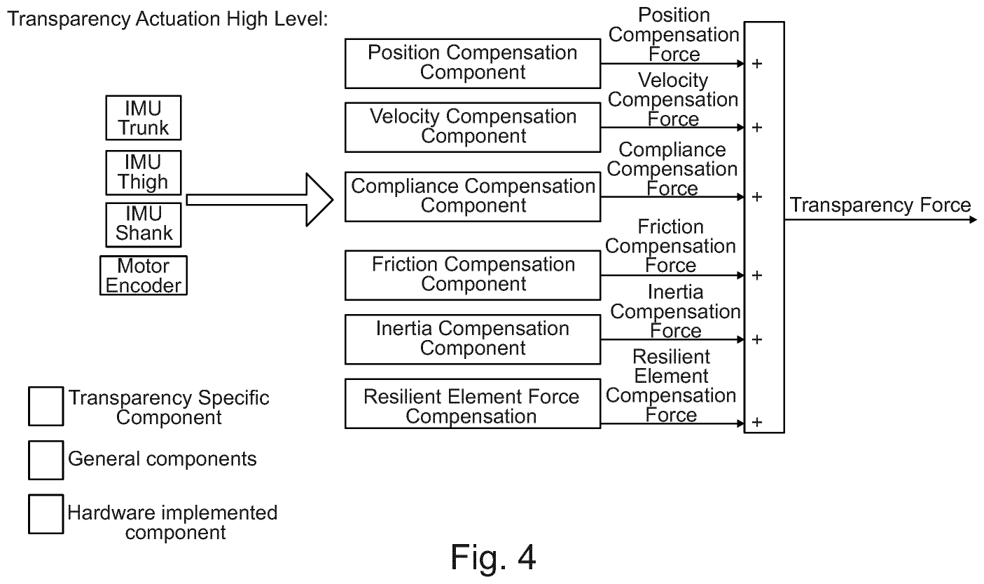

FIG. 4 shows a schematic high level block diagram for modeling a transparency behavior of a wearable active assisting device according to the present invention;

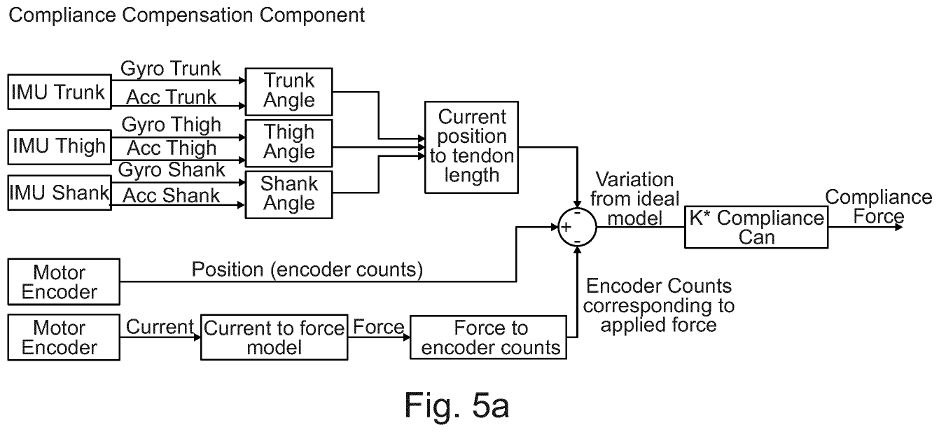

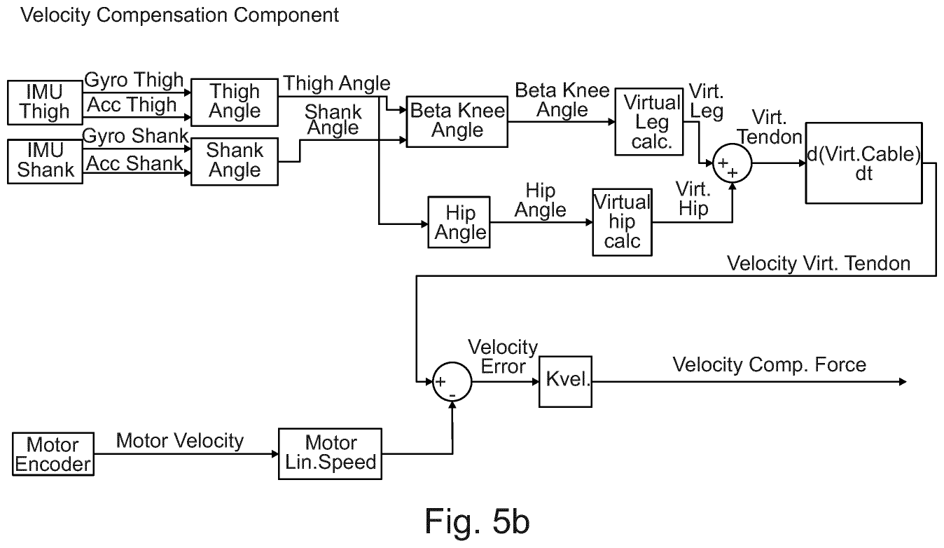

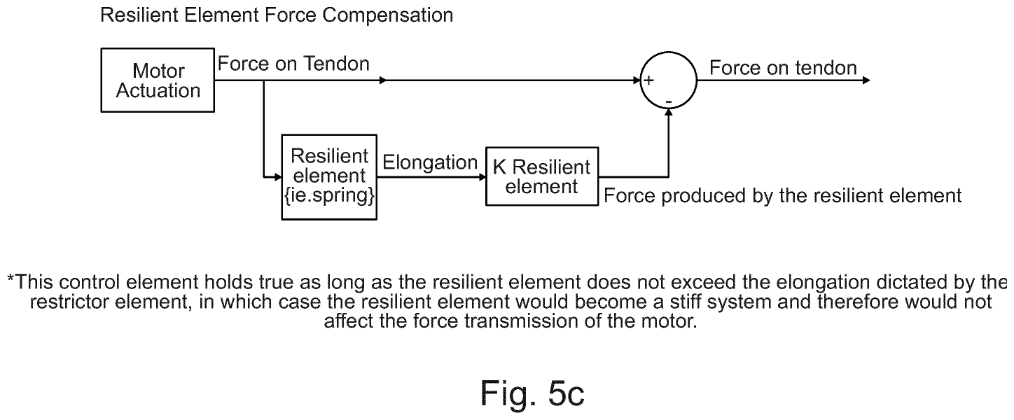

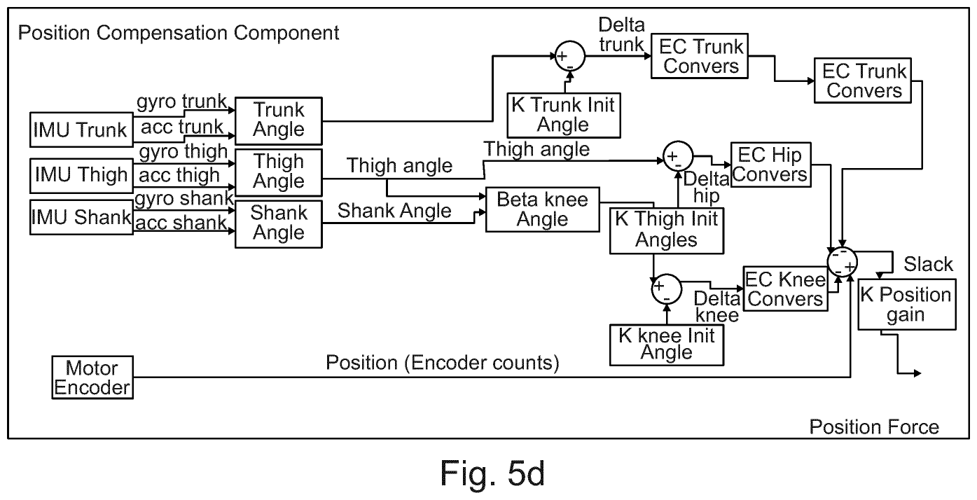

FIGS. 5a-d show model components in more detail, namely a compliance compensation component;

FIG. 5b a velocity compensation component;

FIG. 5c a resilient element force compensation; and

FIG. 5d a position compensation component;

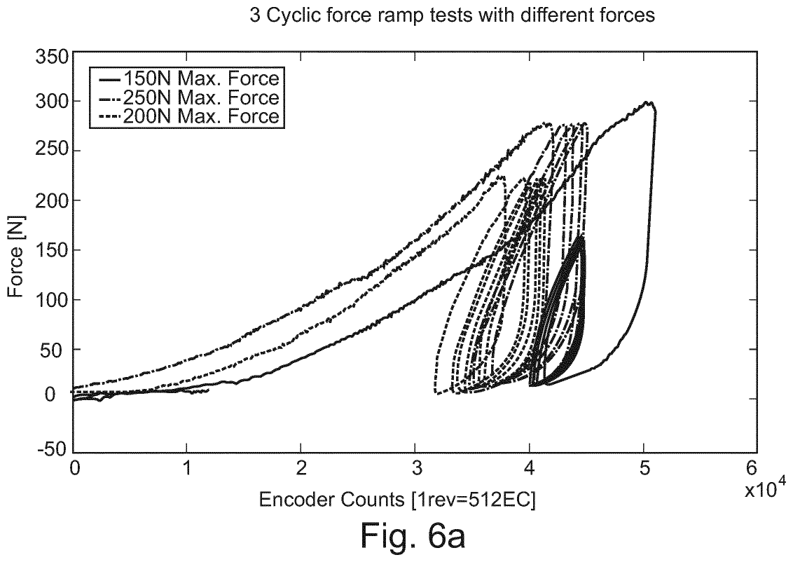

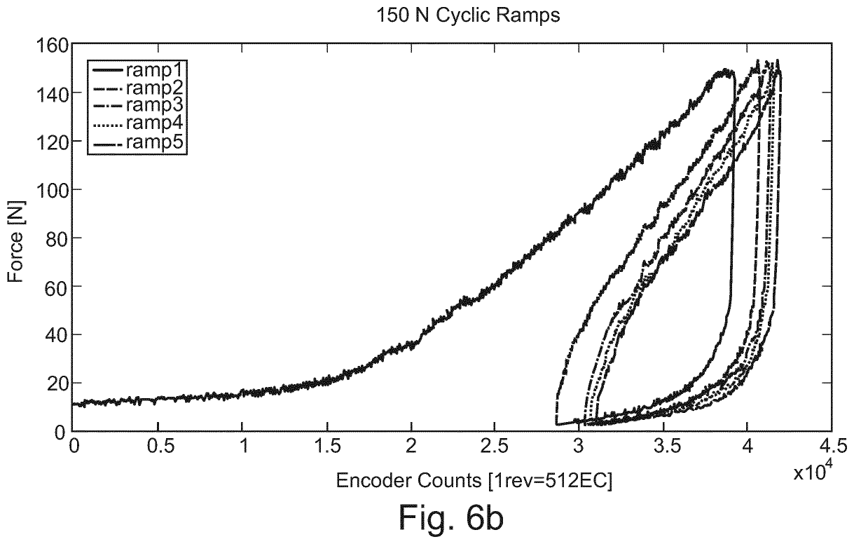

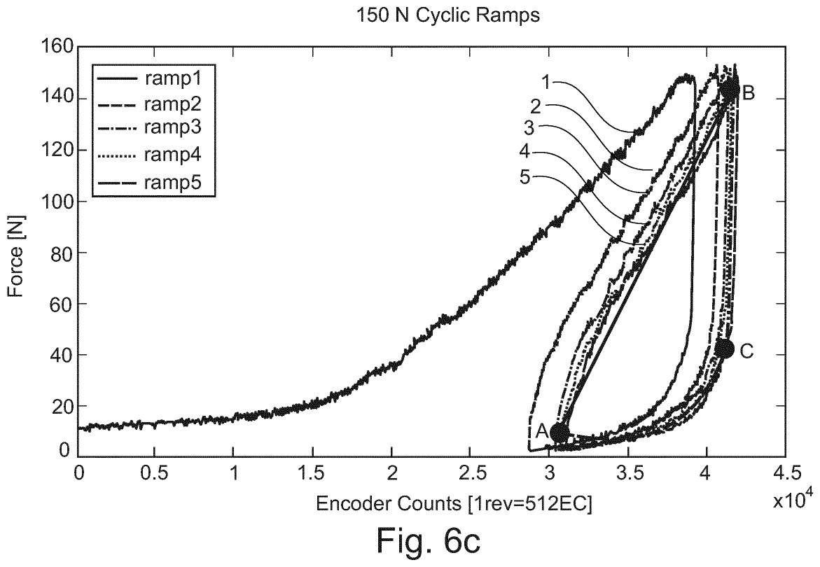

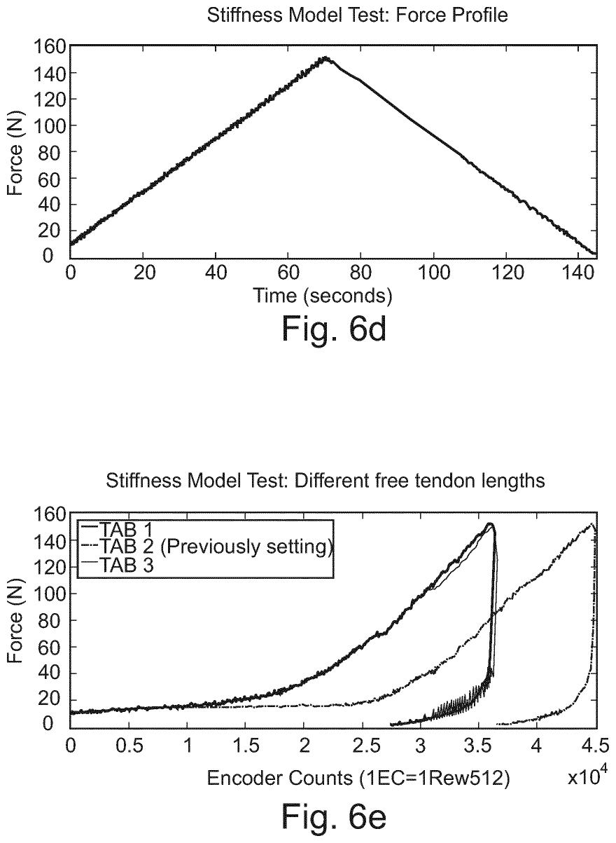

FIG. 6a a force-tendon length relationship for different forces ramped up repeatedly in a cyclic manner;

FIG. 6b shows in more detail of the force-tendon length-relation for a fixed force and repeated force ramps; note that the rather than the tendon length, the encoder counts of a rotating actuator are indicated;

FIG. 6c the force-tendon length-relation of FIG. 6 with an average behavior obtained after repeated cycling;

FIG. 6d a demonstration showing that a force applied to a tendon can be precisely controlled;

FIG. 6e the force-tendon length relation for different postures;

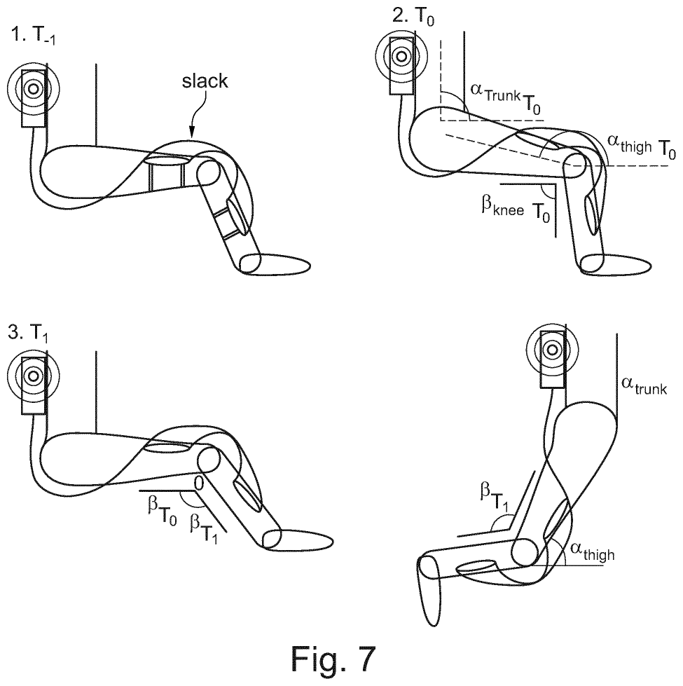

FIG. 7 illustrates that different tendon lengths are needed for minimum support and/or transparency mode in different postures;

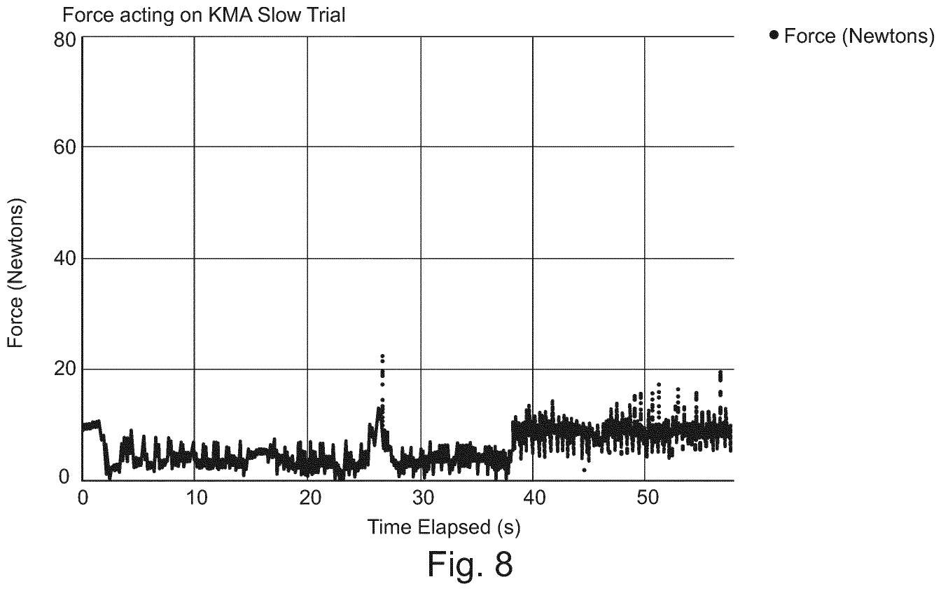

FIG. 8 shows the forces acting on the knee-moment-arm during a transparency mode when moving slowly showing that only minimum forces are applied during transparency mode.

According to FIG. 1, a wearable active assisting device 1 comprises a motor 2 actuatable and used to provide assistance to a limb 3 of a user 4, the motor 2 being coupled to the limb 3 via at least one force transmission element 5 to be elongated or shortened by the motor and a control 6 having an input for signals 7a, 7b, 7c, 7d from a plurality of sensors 8a,8b, 8c, 8d, the controller having a signal processing stage for processing input signals 7a-7d from the plurality of sensors 8a-8d and an output stage 9 for outputting a motor actuation signal 10 in accordance with the processed sensor signals, wherein the control further has a limb assistance degree selection input 11 for selecting a degree of limb assistance; and wherein the signal processor stage of control 6 is adapted to model an elongation of the at least one force transmission element 5 to be elongated or shortened corresponding to a movement of the user as currently detected using the sensors 8a-8d and to output a motor actuation signal 10 according to a current model elongation of the at least one force transmission element 5 to be elongated or shortened and in response to a selected minimum degree of limb assistance.

It should be noted that while in the embodiment shown, the degree of limb assistance can be selected and the transparency mode implemented by the present invention is used as the minimum degree of limb assistance, this need not necessarily be the case. It is possible to generally keep the assistance precise, in particular intentionally below a maximum degree of assistance, for example in order to reduce the load on the components of the wearable active assisting device such as the motor, battery, tendons and so forth, and to increase longevity of the device.

Still, even in that case, the transparency mode described herein can be considered useful as the transparency mode can be used to define a base elongation starting from which additional assistance is provided. In this manner, for example the overall assistance during a cyclic movement can be more constant. In such a case, the wearable active assisting device might e.g. comprise a motor actuatable to provide joint assistance and coupled to a joint to be actively assisted via at least one force transmission element to be elongated or shortened by the motor; and a control having an input from a plurality of sensors, a signal processing stage for processing the signals and an output stage for outputting a motor actuation signal in accordance with the processed sensor signals; wherein the control comprises a model stage adapted to model the elongation of the at least one force transmission element in a manner keeping assistance at or below a threshold sensible by the user by taking into account both a current movement and a posture of the user as detected by the sensors and an inertia and/or friction of the wearable active assisting device counteracting a movement, and wherein the output stage is adapted to output the motor actuation signal in a accordance with the current modeled elongation and/or limb assistance required.

Now, returning to FIG. 1 and the embodiment shown therein, the user 4 is a human patient requiring a certain degree of assistance but using the wearable active assisting devices also during at least some periods where no active assistance is required.

The force transmission element is a tendon coiled and decoiled on a reel rotated by motor 2 so as to elongate or shorten the force transmission element. This can be seen inter alia in FIG. 7. While the precise way the wearable active assisting device is constructed and the force transmission element is guided along the body of the human user 4 is not shown in FIG. 1, reference can be had to WO 2018/122106 A1 in this respect. Possible although non-restricting examples of wearable active assistive devices in which the invention can be implemented are shown therein.

Also, many details such as the construction from different layers and so forth are shown in the cited document. These are also useful in the present invention, although not absolutely necessary. Thus, while a wearable muscle assisting device having a construction according to the cited document and having sensors according to the cited document and a control that other than as described herein closely corresponds to the cited document is perfectly usable for the present invention, it should be noted that the present invention is not restricted to a wearable active assisting device constructed as in WO2018/122106 A1 and that the basic ideas of the present invention can also be used with wearable active assisting devices having a different construction.

In the embodiment shown, the joints assisted are the knee and hip joints of the user, in particular of the right leg, and a first triaxial accelerometer sensor 8d is provided at the shank and a second triaxial accelerometer is provided at the thigh. Furthermore, angle sensors are provided to indicate the bending angle of the right hip, cf. sensor 8a and of the right knee, cf. sensor 8c. A further angle sensor may be provided at the ankle (not shown in FIG. 1). Different angles are also shown for different postures in FIG. 7.

The force transmission element 5 in the embodiment shown is a tendon made from inextensible material and anchored via a cuff 12 at the shank (cf. FIG. 2). Between the tendon 5 and the cuff 12 a rather resilient helical spring 13 provided. The coil spring 13 is anchored with one end thereof at the cuff 12 and with another end thereof at the end 5a of the tendon 5. Parallel to the helical spring 13 and guided within the coil spring 13 is a rope 14. The length of rope 14 is such that in the rope is slack up to the maximum accepted extension of resilient element 13. Such restriction is of course also implementable with resilient elements other than coil springs, e.g. with rubber bands.

As can be seen in FIG. 3, the length of the tendon running along the leg of the user 3 will depend on the posture of the user, in particular the bending angles of the knee and the hip; furthermore, if the motor is attached at the trunk of the user in a rather high position, the length will also depend on the posture of the trunk itself. It will be understood that the change of the length of tendon 5 will depend inter alia on the path along which the tendon runs close to the human body as implemented by the wearable active assisting device. Depending for example on whether the tendon is guided in front or behind the hip, the length will differ. This of course can be taken into account. This can be in particular done as shown in FIG. 3 by calculating a virtual tendon length by defining a virtual hip and a virtual leg only dependent on the current bending angles, indicated in FIG. 3 as angle α, angle β, angle γ.

Then, it will be obvious to the skilled person that any wearable assistive device will have some mass that also needs to be moved if the user wants to move a limb. For example, when moving the shank, the cuff 12 has to be moved as well as the spring 13, rope 14 together with parts of tendon 5 and so forth. Also, there will be some friction due to the garment-like structure of parts of the wearable active assisting device and the friction within the garment and due to other causes of friction as commonly understood.

Now, if a user is to be provided with zero assistance as the minimum assistance but without being adversely effected by the wearable active assisting device 1, then, the compensation components depicted in FIG. 4 should be taken into account and compensated for such as inertia and friction among other effects and disturbances. Otherwise, the user would have to apply additional forces simply to overcome the additional friction and inertia of the wearable active assisting device. It will also be obvious that the inertia to be overcome may depend on the specific movement. For example, where the shank is to be moved, the inertia to be compensated for will depend on whether the shank is to be supported during the beginning of the swing phase where a high acceleration is needed or during the middle of the swing phase where the velocity basically remains constant for a short time, so that no inertia forces need to be compensated. Also, friction forces may depend on the current velocity and current bending angle. (Note that while for explanation of friction and inertia effects, reference has been made to movement patterns and phases such as stance or swing, determination thereof is not necessary. Rather, determination of the speed of the shank asf. will suffice).

As shown in FIG. 4, the model stage modeling a transparency force will in a preferred embodiment take into account the current posture or position of different parts of the human body, namely the trunk, thigh, shank, and will also take into account the current velocity of the trunk, the thigh and the shank. Then, a friction for each component such as at the trunk, thigh and shank and the respective inertia can be taken into account, as well as a resilient element force component.

It will be obvious that the motor will also contribute to friction and inertia so that further to the sensors such as IMU (inertial measuring units) for the trunk, for the thigh and for the shank respectively, preferably a motor encoder signal should be taken into account as well. Using these signals, the position compensation force, the velocity compensation force, a friction compensation force and an inertia compensation force can be calculated from a position compensation component, the velocity compensation component, a friction compensation component and an inertia compensation component, respectively.

By adding these force components, an overall transparency force is determined that is used to give the user the impression that a wearable active assisting device neither assists nor hinders movement.

As can be seen in FIG. 5a, 5b, 5d in more detail, each IMU comprises gyro-sensors and acceleration sensors, in particular triaxial accelerometer sensors that are respectively designated as gyro thigh, acc thigh, gyro shank, acc shank; gyro trunk, acc trunk; gyro thigh, acc thigh, gyro shank, acc shank. From these sensor signals, a current thigh angle and a current shank angle is calculated which are both used in the determination of the velocity compensation component and in deriving a knee angle and a hip angle. From these angles, a virtual hip angle and a virtual leg length is then calculated based on a non-user-specific model resulting in a suggested length of the virtual tendon.

Repeating this determination over and over, a change in the virtual cable length over time is calculated.

The change of the virtual cable length over time can be compared with the current velocity of the motor derived from a motor encoder signal so as to determine whether or not the current velocity is the correct velocity needed to fully compensate current movement or not. As necessary, the current velocity can then be corrected.

In a similar manner, to determine a position compensation, again the trunk angle, the thigh angle and the shank angle are used and from these a knee angle is now determined. Knee angle and trunk angle are compared with a respective initial angle as the differences determine the change of length. Also, the initial thigh angle is taken into account. In this manner, it can be determined whether the current elongation is correct, or should be increased or should be decreased so as to avoid tensions or slack. Depending on the result of this determination, a force component relating to the current position is determined.

Finally, it is possible and preferred that the compliance of tissue and its effect on the displacement of the tendon when forces are being applied are taken into account in a manner compensating for the tissue compression of the human body when the tendon is applying forces onto it.

It should be noted that with preferred actuators it is possible to determine the winding or unwinding of a tendon using a counter counting rotational angle encoder signals from the actuator and to estimate the force applied to the tendon at the same time, e.g. from the current and/or voltage applied to the actuator. In this manner, a force-tendon length relation can be established and from this, the effects of tissue compression and the like can be estimated. Such relations can be determined repeatedly, taking into account that by tensioning the tendon the path it is guided along the body might slightly change, the textile parts of a wearable active assistive device might glide with respect to each other or change their position somewhat asf. leading to slight changes in the force-tendon length relation. Such variations in the behavior can be deduced for example from FIG. 6b. For the sake of precision, it is noted that in FIG. 6, reference is made to the encoder counts. While the encoder counts are closely related to the tendon length, it will be understood that due to the winding or unwinding of the tendon, a full rotation of an actuating motor will result in a smaller change in case the tendon is fully extended as compared to a case where the full rotation will result in a larger change of tendon length due to the larger diameter of the tendon almost fully wound up. Nonetheless, the general pattern can easily be seen and also, it is easy to correct such effects in a controller, in particular a microprocessor-based controller having certain software modules. Also, it can be seen from FIG. 6c that it is possible to derive an average behavior for increasing or rapidly decreasing forces as shown by the curve AB-BC-CA.

Also, it will be understood that different maximum forces applied will result in different changes, as is obvious for example from FIG. 6a, that increasing a force applied will result in a behavior different from the behavior observed when a force is decreased, as is also obvious from FIG. 6a or 6b, and that the effects described will be different in different postures, compare FIG. 6c. Thus, a general behavior and/or general influence of tissue compression asf. could be modeled; additionally and/or alternatively a behavior could be modeled taking specifically into account whether the force is applied for a first time after significant change of posture or whether application of force is repeated; additionally and/or alternatively a behavior could be modeled taking into account the maximum force currently or previously applied in a given posture; additionally and/or alternatively a behavior could be modeled taking into account a previous posture, in particular the posture(s) immediately preceeding the current posture. FIG. 6d shows a potential force profile to determine the stiffness model of a user. The depicted results in FIG. 6e show additionally the different length of slack in the system that can be compensated for.

The model could be based on average data gathered for a wide range of users and/or could be based on data gathered specifically for a single user, in particular for the specific user and the specific current way the device is worn, taking into account that day-to-day variations may occur and that these variations can be compensated for by establishing or estimating current force-tendon length relations. Note that thus, in a preferred embodiment a model can be determined that correlates forces being applied with measurements of tendon travel so that the system can take into account any potential high pressure points on the user by compensating them, (reeling out cable). It should also be noted that when compressed, the tissue will of course absorb some of the energy and that this can be modeled in a manner treating the tissue as a spring-damper system that by absorbing energy from the force transmitting element helps stabilize potential instabilities during control actions, also improving system response to a safety actuation when a sudden increases of assistance is required.

It should be noted that while the control is adapted to model a force transmission element elongation irrespective of the size and weight of the user, e.g. to determine an elongation necessary for a transparency mode irrespective of the size of a user, an exemption to this can be made with respect to modeling force-tendon length relations as these can be determined easily as is obvious from the above.

Now, while it would be possible to model the exact behavior of the wearable active assisting device for a specific user having a specific size, this would usually require a number of measurements that frequently would have to be repeated over and over again, for example because the legs of a patient are initially swollen following an accident, the swelling slowly decreasing over time.

Therefore, it is desirable to use the resilient element as explained with respect to FIG. 2 to use general parameters and to elongate or shorten the tendon only to a precision such that the spring 13 is not fully extended during a transparency mode. Only when actual assistance is needed, for example because a patient becomes exhausted, the tendon 5 will be shortened so much that element 14 is no longer slack. As the distance the tendon 5 has to be reeled in will be extremely small during transparency mode of the present invention, active assistance can be provided almost immediately and without causing a shock or jerk to the limb supported.

1. A wearable active assisting device comprising:
an actuator;
at least one force transmission element, wherein the at least one force transmission element is configured to be elongated or shortened by the actuator, wherein the actuator is configured to provide a limb assistance and is configured to be coupled to a limb to be actively assisted via the at least one force transmission element;
a plurality of sensors;
a control comprising an input for receiving signals from the plurality of sensors;
a signal processing stage for processing input signals from the plurality of sensors; and
an output stage for outputting a motor actuation signal in accordance with the input signals processed in the signal processing stage;
wherein the control further comprises a limb assistance degree selection input for selecting a degree of limb assistance; and
wherein the signal processing stage is adapted to continuously model an elongation of the at least one force transmission element to be elongated or shortened corresponding to a movement or posture currently detected by the plurality of sensors to output a continuous actuator actuation signal according to a modeled elongation of the at least one force transmission element to be elongated or shortened and in response to a selected minimum degree of limb assistance.

2. A wearable active assisting device according to claim 1, wherein a system response is determined according to physical characteristics of a human body.

3. The wearable active assisting device according to claim 1, wherein the control is adapted to model a force transmission element elongation.

4. The wearable active assisting device according to claim 1, wherein the plurality of sensors comprises one or more gyro-sensors, accelerometer sensors, magnetometer sensors, stretchable sensors, kinematic sensors, angle sensors, or a combination of any thereof.

5. The wearable active assisting device according to claim 4, wherein the plurality of sensors comprises at least one gyro-sensor and/or accelerometer sensor, wherein each of the plurality of sensors is configured to be positioned at each of a plurality of limbs or joints.

6. The wearable active assisting device according to claim 5, wherein a current force transmission element elongation is determined in response to sensor signals.

7. The wearable active assisting device according to claim 1, wherein the control is adapted to model a force transmission element elongation independent of any force or tension indicative signals indicative of a force and/or tension in the at least one force transmission element detected from the plurality of sensors.

8. The wearable active assisting device according to claim 1, wherein the control is adapted to model an elongation of the at least one force transmission element to be elongated or shortened in a manner taking friction of the at least one force transmission element and/or inertia into account.

9. The wearable active assisting device according to claim 1, further comprising a resilient elastic element provided in series with the force transmission element to be elongated or shortened, the resilient elastic element configured to be positioned between the limb to be actively assisted and the actuator, and wherein a restrictor for restricting the elongation of the resilient elastic element to a maximum allowed elongation is provided.

10. The wearable active assisting device according to claim 9, wherein the resilient elastic element has a modulus of resilience such that for a maximum residual force accepted in a selected minimum degree of limb assistance, the resilient elastic element is elongated by no more than the maximum allowed deviation between a standardized model and an extension correction.

11. The wearable active assisting device according to claim 1, wherein the at least one force transmission element to be elongated or shortened comprises a force transmission element, wherein a reel is provided to reel or unreel the force transmission element when shortening or elongating, and wherein the actuator is a step motor or a brushless motor.

12. The wearable active assisting device according to claim 1, wherein the force transmission element is guided in a slack sheath.

13. (canceled)

14. The wearable active assisting device according to claim 1, wherein in a minimum degree of assistance, the control is adapted to maintain the elongation at a residual force.

15. The wearable active assisting device according to claim 14, wherein the residual force is smaller than 30 N.

16. The wearable active assisting device according to claim 15, wherein the residual force is smaller than 20 N.

17. The wearable active assisting device according to claim 16, wherein the residual force is smaller than 10 N.

18. The wearable active assisting device according to claim 14, wherein the residual force induced by the force transmission element is larger than 0.5 N.

19. The wearable active assisting device according to claim 18, wherein the residual force induced by the force transmission element is larger than 1 N.

20. The wearable active assisting device according to claim 18, wherein the residual force induced by the force transmission element is between 0.5 N and 5 N.

21. The wearable active assisting device according to claim 14, wherein the residual force induced by the force transmission element occurs for at least for a part of a movement detected by the plurality of sensors.

22. The wearable active assisting device according to claim 21, wherein the residual force induced by the force transmission element occurs for at least 50% of a cyclic movement.

23. The wearable active assisting device according to claim 22, wherein the residual force induced by the force transmission element occurs for at least 66% of a cyclic movement.

24. The wearable active assisting device according to claim 23, wherein the residual force induced by the force transmission element occurs for at least 75% of a cyclic movement.

25. The wearable active assisting device according to claim 1, wherein the control is adapted to model the elongation such that when transitioning from a selected minimum degree of assistance to a degree of assistance higher than the minimum degree, a force transmission element slack of no more than 10 cm needs to be overcome by reeling.

26. The wearable active assisting device according to claim 25, wherein a force transmission element slack of no more than 7 cm needs to be overcome by reeling.

27. The wearable active assisting device according to claim 25, wherein a force transmission element slack of no more than 5 cm needs to be overcome by reeling.

28. The wearable active assisting device according to claim 1, wherein the active assisting device is adapted to assist in leg activity and the plurality of sensors comprises at least one gyro- and accelerometer sensor configured to be positioned at each of a plurality of limbs or joints.

29. The wearable active assisting device according to claim 28, wherein a current force transmission element elongation is determined in response to sensor signals.

30. The wearable active assisting device according to claim 1, wherein in response to the sensor signals the model is adapted to identify a current intended movement from the sensor signals, to determine a phase in the current movement identified, to model a change of the force transmission element elongation according to the expected progress of the current movement, and to output a motor actuation signal in response to the modeled elongation.

31. The wearable active assisting device according to claim 1, wherein the control is adapted to identify an activity as a current movement and wherein the control is adapted to determine a phase in the current activity as a stance phase or swing phase and/or determine foot-ground-contact and/or determine a phase in stair climbing and/or ascending and/or walking, uphill or downhill and/or sitting transitions.

32. A control for a wearable active assisting device,
the wearable active assisting device comprising an actuator, the actuator configured to be actuatable to provide limb assistance and configured to be coupled to a limb to be actively assisted via at least one force transmission element to be elongated or shortened by a motor;
the control comprising
an input for signals from a plurality of sensors,
a signal processing stage for processing the signals,
and
an output stage for outputting a motor actuation signal in accordance with the processed sensor signals;
and wherein the control comprises a model stage adapted to model:
the elongation of the at least one force transmission element in a manner keeping assistance at or below a threshold,
a current movement and/or posture of a user detected by the sensors, and
inertia and/or friction of the wearable active assisting device,
and wherein the output stage is adapted to output the motor actuation signal in accordance with the current modeled elongation and a limb assistance required.

33. The control according to claim 32, wherein the control is adapted to model an elongation component in a continuous manner without reference to a predefined force/assistance profile.

34. The control according to claim 33, wherein the control is further adapted to determine an additional elongation component to be simultaneously applied in response to a detected and/or identified movement, wherein the additional elongation is simultaneously applied to the transparency mode elongation.

35. The control according to claim 34, wherein the detected and/or identified movement is a walking movement, a stair climbing movement, and/or a sitting transition movement.
